(19) United States
(12) Patent Application Publication
(10) Pub. No.: US 2018/0220925 A1
(43) Pub. Date: Aug. 9, 2018

(54) DETERMINING A POSITION OF A SUBJECT UNDER EXAMINATION DURING IMPLEMENTATION OF A MEDICAL IMAGING PROCEDURE

(71) Applicants: Lars Lauer, Neunkirchen (DE); Steffen Schröter, Fürth (DE)

(72) Inventors: Lars Lauer, Neunkirchen (DE); Steffen Schröter, Fürth (DE)

(21) Appl. No.: 15/887,532

(22) Filed: Feb. 2, 2018

(30) Foreign Application Priority Data
Feb. 3, 2017 (DE) 102017201750.3

(51) Int. Cl. A61B 5/055; A61B 5/00; G01R 33/565; G01B 11/00

(52) U.S. Cl. CPC A61B 5/0555; A61B 5/7207; G01R 33/56509; A61B 2560/04; A61B 5/0077; A61B 2562/0233; G01B 11/002

(57) ABSTRACT

A system and method are provided for determining a position of a subject under examination during implementation of a medical imaging procedure. A radiation generating unit generates optical radiation that is used to illuminate the subject under examination. The subject under examination blocks the generated optical radiation to produce a shadow. The shadow is detected by an optical detection unit and used by a position determination unit to determine a position of the subject under examination.

### CROSS REFERENCE TO RELATED APPLICATIONS

This application claims the benefit of DE 102017201750.3, filed on Feb. 3, 2017, which is hereby incorporated by reference in its entirety.

### FIELD

Embodiments relate to systems and methods for determining a position of a subject under examination during implementation of a medical imaging procedure

### BACKGROUND

It is useful during implementation of a medical imaging procedure, for example magnetic resonance imaging or computed tomography, to determine a position and/or a motion of a subject under examination, for example a human or animal patient, that is examined by a suitable medical imaging apparatus.

US 20150164440 A1 discloses a method for setting an acquisition region for medical imaging using a medical imaging apparatus, in which method a 3D camera is used to generate an image of a patient on a patient couch.

WO 2015128108 A1 discloses a method for adjusting an X-ray unit. Image acquisition units such as cameras, for instance, are used to capture optical image data.

US 20150077113 A1 discloses a medical imaging apparatus and a method for determining a position and/or a motion of a patient during a medical imaging examination. A camera captures motion data relating to a motion of a patient during a magnetic resonance examination.

Motion and/or position data relating to the patient and captured during the medical imaging examination may be used to correct medical image data obtained by the medical imaging procedure in respect of a motion of the patient.

Conventional motion correction methods may use markers that are fixed to the patient, to achieve an adequate level of accuracy. One or more cameras are used, for example, for such motion correction methods. In order to take into account skin movements, three or more markers may be attached that are detected by the cameras. Attaching and removing the markers impedes the workflow and may result in a negative impact on patient comfort.

### SUMMARY AND DESCRIPTION

The scope of the present invention is defined solely by the appended claims and is not affected to any degree by the statements within this summary. The present embodiments may obviate one or more of the drawbacks or limitations in the related art.

Embodiments provide a method to dispense with using markers when determining a position, for example, a motion, of a subject under examination during implementation of a medical imaging procedure.

A method for determining a position of a subject under examination during implementation of a medical imaging procedure is provided. A radiation generating unit generates optical radiation that is used to illuminate the subject under examination. The subject under examination blocks the generated optical radiation to produce a shadow. The shadow, e.g. the position and/or shape thereof, is detected by an optical detection unit and used by a position determination unit to determine a position of the subject under examination.

The optical radiation may be electromagnetic radiation with a wavelength in a vacuum of between 10 nm and 1 mm, for example, between 100 nm and 1000 nm. The optical radiation may include infrared radiation and/or ultraviolet radiation in addition to the visible light. The wavelength region, e.g. the wavelength region of visible light, may be used as there are a large number of configured and low-cost radiation generating units and optical detection units available for the region. In addition, a wavelength that is not too long allows a sharp cast shadow and hence exact determination of the position of the subject under examination. Moreover, visible light may also be used to have a positive impact on the wellbeing of the subject under examination while the medical imaging procedure is being performed.

The radiation generating unit includes at least one radiation source. A radiation source may include, for example, one or more light sources such as, for example, light-emitting diodes (LEDs) and/or incandescent lamps and/or gas discharge lamps and/or induction lamps.

The optical detection unit includes at least one detection module, for example, an optical sensor that is configured to detect the optical radiation generated by the radiation generating unit and possibly reflected by a reflection surface. The optical detection unit is sensitive in the wavelength region of the generated optical radiation. The optical detection unit includes, for example, one or more cameras, that includes, for example, one or more CCD sensors (Charge Coupled Devices) and/or CMOS sensors (Complementary Metal-Oxide-Semiconductor sensors).

In an embodiment, the generated optical radiation does not illuminate the entire subject under examination but instead illuminates just a part of the subject under examination. Only the illuminated part of the subject under examination rather than the entire subject under examination contributes to producing a shadow from the generated optical radiation.

The subject under examination is illuminated such that the subject casts a shadow that is detected by the optical detection unit. A projected image of the subject under examination that is located in the direction of propagation of the generated optical radiation, is detected, e.g. that is produced by the shadowing. The optical detection unit detects a reflected portion of the generated optical radiation, e.g. that delimits the shadow. A line that delimits the shadow may also be referred to as a light/dark boundary and/or shadow boundary and/or silhouette. The line shows an outline and/or silhouette of the subject under examination.

A shadow may refer, for example, to a region that, behind the subject under examination from the perspective of a radiation source of the radiation generating unit, is unaffected by the radiation source of the radiation generating unit and hence is dark, e.g. a surface that is not illuminated by the radiation source.

The shadow produced may replace conventional markers avoiding the disadvantages associated with attaching markers. A shadow may include sharp edges that may be used to determine the position of the subject under examination.

In a further embodiment, detecting the shadow by an optical detection unit is repeated successively. For example, the shadow is detected at different successive points in time, so that a motion of the subject under examination may be determined.

Determining a position of the subject under examination from the detected shadow by a position determination unit may also include determining a motion of the subject under examination. Embodiments may provide motion-corrected medical images without disrupting the workflow.

In a further embodiment, the optical radiation is pulsed, e.g. flashes. Generating the optical radiation may include, for example, generating pulsed radiation, e.g. light pulses and/or light flashes. The radiation generating unit may include one or more stroboscopes and/or radiation source operating in the manner of a stroboscope.

The pulsed optical radiation may be generated, for example, by the radiation generating unit generating optical radiation that is continuous over time and is chopped into pulsed optical radiation by a stop and/or a chopper and/or a diaphragm shutter and/or an optical shutter. The optical radiation may be generated in pulsed form from the start, e.g. by a pulsed current flow through a light-emitting diode.

The radiation generating unit may emit, for example, pulsed optical radiation at regular time intervals. For example, in a dark environment, any movements of the subject under examination appear chopped as a sequence of stationary images.

Interference sources that have a different frequency from the generated pulsed optical radiation may be identified as external and may be excluded. A lock-in measurement technique may be used, e.g. using a lock-in amplifier, to detect the shadow. allowing measurements that have a high signal-to-noise ratio.

The optical radiation may include a pulse frequency of between 0.1 Hz and 10 kHz, for example, between 1 Hz and 1 kHz, i.e. the time period between two radiation pulses may be between 0.1 ms and 10 s, in particular between 1 ms and 1 s.

Thus, the illumination of the subject under examination and/or generation of the shadow may be performed in a pulsed manner. The optical detection unit may detect the shadow for example continuously and/or synchronously with the pulsed radiation, e.g. by performing detection of the silhouettes only at the points in time when the shadows are being cast.

In an embodiment, the optical radiation includes a pulse frequency of greater than 60 Hz. A person as the subject under examination usually perceives a radiation generating unit that is emitting light at such a pulse frequency as a steady, dimmed light source. The person may find such a visual impression pleasant.

In a further embodiment, the shadow includes a pointed contour. A pointed contour may refer to a contour in the form of a narrow taper. For example, the pointed contour may be described by three points connected by lines that include an angle smaller than 90°.

For example, illuminating a nose tip of a subject under examination is suitable for producing a shadow that has a pointed contour. Illuminating the subject under examination by the generated optical radiation may involve illuminating a nose and/or other protruding parts of the body of the subject under examination.

In a further embodiment, the generated optical radiation illuminates the subject under examination from different directions. The shadow may be cast in different directions.

For example, the nose tip of a patient may be illuminated from a first direction in relation to the patient, thereby casting a first shadow opposite the first direction. In addition, the nose tip of the patient may also be illuminated from a second direction that differs from the first direction, thereby casting a second shadow opposite the second direction.

Determining a position of the subject under examination from the detected shadows may be performed on a broad information basis that may include redundancies. The position may be determined reliably.

For example, the generated optical radiation may illuminate the subject under examination successively and/or simultaneously from different directions.

For example, at a first point in time, the subject under examination is illuminated from a first direction, and at a second point in time from a second direction that differs from the first direction. The subject under examination may be additionally illuminated from a third direction both at the first and at the second point in time.

Successive illumination, e.g. illumination performed in succession, from different directions provides optimization of the signal-to-noise ratio of the shadow detection by an optical detection unit. Simultaneous illumination from different directions provides an increase in the acquired information density.

In a further embodiment, the subject under examination is illuminated from a first direction by first generated optical radiation, and from a further direction, that differs from the first direction, by further generated optical radiation. The first optical radiation includes a first pulse frequency and the further optical radiation includes a further pulse frequency. The first pulse frequency differs from the further pulse frequency.

Interference sources that include a different frequency from the first and/or further pulse frequency may be identified as external and may be excluded.

The subject under examination may be illuminated at different pulse frequencies from additional directions apart from the first direction and the further direction, e.g. the principle is applied to more than two directions.

For example, a first radiation source provides continuous illumination, e.g. the pulse frequency is zero, a second radiation source provides pulsed illumination at 100 Hz and a third radiation source provides pulsed illumination at 120 Hz.

The illumination sources may differ in terms of pulse lengths. For example, the subject under examination is illuminated from a first direction by first generated optical radiation, and from a further direction by further generated optical radiation. The first optical radiation includes pulses of a first pulse length. The further optical radiation includes pulses of a further pulse length that differ from the first pulse length.

The pulse lengths may be relative and/or absolute pulse lengths. A relative pulse length may refer to a pulse length that is specified, e.g. as a percentage, in relation to a time period between two successive light pulses. An absolute pulse length may refer to a pulse length that is specified as an absolute time value, e.g. in seconds.

An effective light intensity may be modulated so that it is possible to distinguish between the shadows from different light sources. External light sources may thus be eliminated.

The subject under examination may be illuminated using different pulse lengths from additional directions apart from the first direction and the further direction, e.g. the principle is applied to more than two directions. Also, a combination of different pulse frequencies and pulse lengths may be used.

For example, a first radiation source provides continuous illumination, and a second radiation source and third radiation source provide pulsed illumination at 100 Hz. The second radiation source is on for 50% and off for 50%, whereas the third radiation source is on for 10% and off for 90%, resulting in a different illumination pattern for each radiation source.

In an embodiment, the illumination from different directions may be offset in time, resulting in, for example, the illumination from the first direction occurring in a different time window than the illumination from the further direction.

In an embodiment, the direction from which the generated optical radiation illuminates the subject under examination is determined from at least one already detected shadow.

The determination may be performed by an analysis unit. For example, the analysis unit analyzes the at least one already detected shadow, and from the analysis derives the future illumination direction from which relevant information for determining a future position of the subject under examination from a shadow detected in the future.

For a rotational motion of the subject under examination, the determination may identify when the illumination of the subject under examination from certain directions does not produce an evaluable shadow, and when other illumination directions may be used.

The radiation generating unit may include a plurality of radiation sources that are arranged in different directions, for example, on different sides, relative to the subject under examination. The subject under examination may thereby be illuminated from different directions.

In an embodiment, the radiation generating unit may include two or more radiation sources. For example, a first radiation source is arranged above a patient head, a second radiation source below the head, a third radiation source to the right of the head and a fourth radiation source to the left of the head.

In an embodiment, the radiation generating unit may include a plurality of radiation sources that are arranged in different directions on different sides relative to a support surface, for example, to a midpoint of the support surface.

Vectors directed from the midpoint of the support surface to the radiation sources may include angles of at least 360°/(4*number of radiation sources), for example at least 360°/(2*number of radiation sources) resulting in good spatial coverage by the radiation sources and providing the position of the subject under examination to be determined reliably.

In an embodiment, the radiation generating unit includes at least one radiation source that is configured to emit the generated optical radiation in different directions.

The radiation generating unit includes, for example, an electronic and/or mechanical tilting mechanism, that may be used to alter the emission direction of the at least one radiation source. The direction of the cast shadow may thereby be adjusted.

In an embodiment, the optical detection unit detects the shadow from different directions, for example, simultaneously. The subject under examination may thereby be viewed from different perspectives and/or angles, with the result that shadows may be detected more reliably and more accurately.

The optical detection unit may include a plurality of detection modules, e.g. a plurality of cameras, that are arranged on different sides relative to the subject under examination.

The detection unit includes, for example, four detection modules. A first detection module is arranged above a patient head, a second detection module below the head, a third detection module to the right of the head and a fourth detection module to the left of the head.

In an embodiment, the detection unit includes a plurality of detection modules, that are arranged in different directions on different sides relative to a support surface, for example to a midpoint of the support surface.

Vectors directed from the midpoint of the support surface to the detection modules may include angles of at least 360°/(4*number of detection modules), for example at least 360°/(2*number of detection modules), that provide good spatial coverage by the detection modules and allow the position of the subject under examination to be determined reliably.

In an embodiment, a medical imaging apparatus is provided that includes a radiation generating unit, an optical detection unit and a position determination unit. The medical imaging apparatus is configured to perform a method for determining a position of a subject under examination during implementation of a medical imaging procedure.

Features, advantages or alternative embodiments mentioned for the method may also be applied to the medical imaging apparatus for example, and vice versa. The corresponding functional features of the method are embodied in this case by corresponding physical modules, in particular by hardware modules.

The medical imaging apparatus may be, for example, an apparatus for carrying out magnetic resonance imaging (MRI), computed tomography (CT) and/or positron emission tomography (PET). Position correction, for example, motion correction, may be employed in the production of MRI images due to the relatively long acquisition time.

In one embodiment of the medical imaging apparatus, the radiation generating unit includes at least one radiation source, and the detection unit includes at least one detection module. The at least one radiation source and the at least one detection module are arranged in a shared housing. A radiation source and a detection module may be integrated into one component providing a space-saving configuration.

In an embodiment, a computer program product is provided a program that may be loaded directly into a memory of a programmable processing unit of a position determination unit of a medical imaging apparatus. The computer program product may include programming, e.g. libraries and auxiliary functions, to perform a method for determining a position of a subject under examination during implementation of a medical imaging procedure when the computer program product is executed in a position determination unit. The computer program product may include software containing a source code, that may be compiled and linked or interpreted, or an executable software code, that may be loaded into the position determination unit. The method may be performed reproducibly and robustly by the computer program product. The computer program product is configured such that the computer program product may use the position determination unit to perform the method. The position determination unit advantageously may include, for example, a suitable RAM or a suitable logic unit, in order to be able to perform the respective method efficiently.

### BRIEF DESCRIPTION OF THE FIGURES

Further advantages, features and details appear in the embodiments described below and follow from the drawings. Corresponding parts are denoted by the same reference signs in all the figures.

FIG. 1 depicts a schematic diagram of a magnetic resonance machine as an example of a medical imaging apparatus.

FIG. 2 depicts a block diagram of a method for determining a position of a subject under examination according to an embodiment.

FIG. 3 depicts a schematic diagram of a time sequence of actions for illuminating a subject under examination and actions for detecting a resultant shadow according to an embodiment.

FIG. 4 depicts a schematic diagram of a time sequence of actions for illuminating a subject under examination from a plurality of directions according to an embodiment.

FIGS. 5 and 6 depict diagrams for the production and detection of a shadow according to an embodiment.

### DETAILED DESCRIPTION

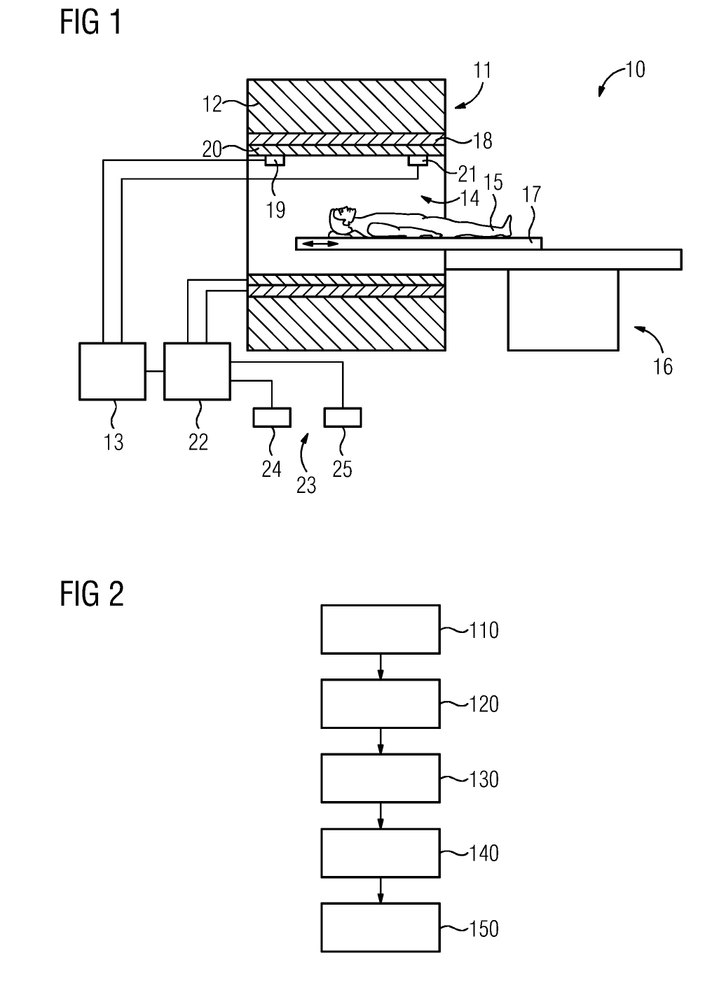

FIG. 1 depicts a magnetic resonance machine 10 as an example of a medical imaging apparatus. The magnetic resonance machine 10 includes a magnet unit 11 that includes a main magnet 12 for producing a powerful main magnetic field 13 that may be constant over time. The magnetic resonance machine 10 also includes a patient receiving zone 14 for accommodating a subject under examination 15, e.g. a patient. In an embodiment, the patient receiving zone 14 is shaped as a cylinder and is enclosed in a circumferential direction cylindrically by the magnet unit 11. The patient receiving zone 14 may include a different configuration. The subject under examination 15 may be moved into the patient receiving zone 14 by a patient support apparatus 16 of the magnetic resonance machine 10. The patient support apparatus 16 includes a patient couch 17 that is configured to be able to move inside the patient receiving zone 14.

The magnet unit 11 further includes a gradient coil unit 18 for generating magnetic field gradients that are used for spatial encoding during imaging. The gradient coil unit 18 is controlled by a gradient control unit 19 of the magnetic resonance machine 10. The magnet unit 11 also includes an RF antenna unit 20 that may be configured as a body coil that is permanently built into the magnetic resonance machine 10. The RF antenna unit 20 is configured to excite nuclear spins that are established in the main magnetic field 13 produced by the main magnet 12. The RF antenna unit 20 is controlled by an RF antenna control unit 21 of the magnetic resonance machine 10 and radiates high-frequency magnetic resonance sequences into an examination space that is formed by a patient receiving zone 14 of the magnetic resonance machine 10. The RF antenna unit 20 is also configured to receive magnetic resonance signals.

The magnetic resonance machine 10 includes a system control unit 22 for controlling the main magnet 12, the gradient control unit 19 and the RF antenna control unit 21. The system control unit 22 centrally controls the magnetic resonance machine 10, for example by implementing a predetermined imaging gradient echo sequence. In addition, the system control unit 22 includes an analysis unit (not shown in further detail) for analyzing medical image data acquired during the magnetic resonance examination. Furthermore, the magnetic resonance machine 10 includes a user interface 23 that is connected to the system control unit 22. Control information such as imaging parameters, for example, and reconstructed magnetic resonance images may be displayed to medical personnel on a display unit 24, for example on at least one monitor, of the user interface 23. Moreover, the user interface 23 includes an input unit 25 that may be used by the medical operating personnel to enter data and/or parameters during a measurement process.

The magnetic resonance machine 10 also includes a radiation generating unit 19, an optical detection unit 21 and a position determination unit 13. The position determination unit 13 may include a programmable processing unit including one or more processors and a memory. By the position determination unit 13, a computer program product (not shown in further detail here) may be used to perform a method for determining a position of a subject under examination 15 during implementation of a medical imaging procedure. The computer program product includes a program and may be loaded directly into the memory of the programmable processing unit of the position determination unit 13. The method is performed when the program is executed in the processing unit of the position determination unit 13.

The radiation generating unit 19 is configured to generate optical radiation. The radiation generating unit 19 may include one or more radiation sources. The one or more radiation sources may be configured to generate visible light, infrared radiation and/or ultraviolet radiation. The one or more radiation sources may include, for example, one or more light sources such as, for instance, light-emitting diodes (LEDs) and/or incandescent lamps and/or gas discharge lamps and/or induction lamps.

The optical detection unit 21 is configured to detect the optical radiation. The optical detection unit 21 includes, for example, one or more detection modules such as cameras.

The radiation generating unit 19 and the detection unit 21 may also be arranged in a shared housing, unlike the representation in FIG. 1.

The position determination unit 13 is configured to control the radiation generating unit 19 and to receive detection signals from the optical detection unit 21. The position determination unit 13 is also configured to determine a position of the patient 15 from the detection signals.

FIG. 2 depicts a method for determining a position of a subject under examination 15 during implementation of a medical imaging procedure.

At act 110, optical radiation is generated by a radiation generating unit 19. The optical radiation may be generated, for example, in a pulsed manner. The radiation generating unit 19 may generate light flashes, for example, as the radiation pulses. At act 120, the subject under examination 15 is illuminated by the generated optical radiation.

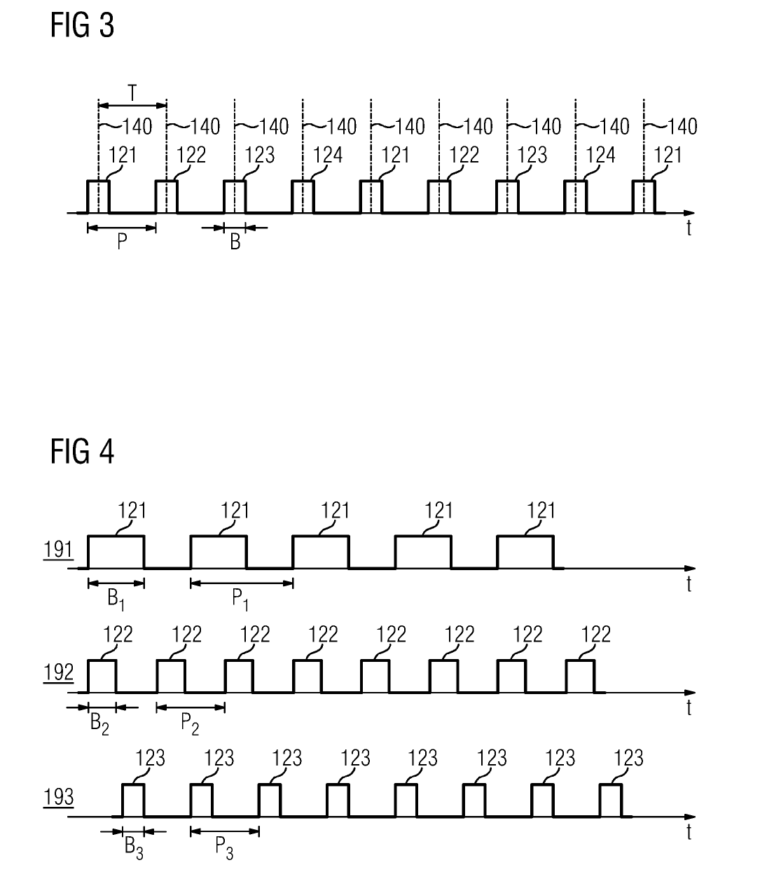

As depicted in FIG. 3, radiation pulses 121, 122, 123, 124 of pulse length B may be generated at regular time periods P, and are used to illuminate the subject under examination 15. The optical radiation may include a pulse frequency f of greater than 60 Hz, i.e. f=1/P>60 Hz. In this frequency range, a human subject under examination 15 perceives the radiation pulses as steady, dimmed light, so that the subject under examination 15 finds the illumination pleasant.

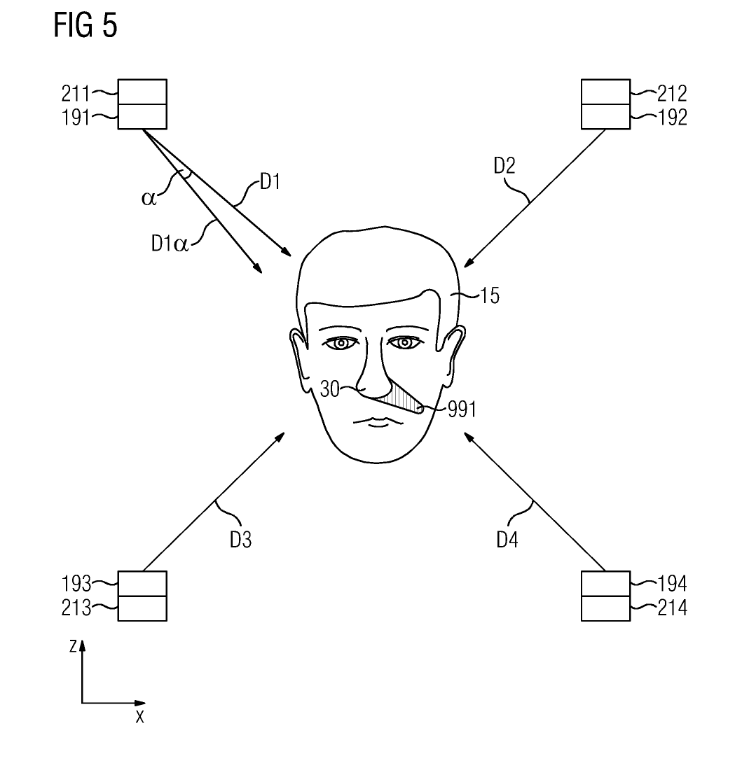

FIG. 4 depicts over time an example of a sequence of actions for illuminating from different directions. In the example, 191, 192, 193 each constitute a radiation source representing illumination from a specific direction, e.g. the radiation source 191 illuminates the subject under examination 15 from a first direction using first generated optical radiation 121, the radiation source 192 illuminates the subject under examination 15 from a second direction using second generated optical radiation 12, and the radiation source 193 illuminates the subject under examination 15 from a third direction using third generated optical radiation 123.

The first optical radiation has a first pulse frequency f₁=1/P₁, the second optical radiation has a second pulse frequency f₂=1/P₂, and the second optical radiation has a second pulse frequency f₃=1/P₃. The first pulse frequency f₁ differs from the second pulse frequency f₂. The second pulse frequency f₂ is in the example equal to the third pulse frequency f₃, although the second pulse frequency f₂ and the third pulse frequency f₃ may also be different.

Moreover, in the example the first optical radiation 121 includes pulses of a first pulse length B₁, the second optical radiation 122 includes pulses of a second pulse length B₂, and the third optical radiation 123 includes pulses of a third pulse length B₃. The pulse length may also be specified as a proportion in relation to the associated time period between two pulses, so for instance: B₁/P₁=50%, B₂/P₂=35%, B₃/P₃=25%.

The signal-to-noise ratio may be improved by modulating the illumination with different pulse frequencies and/or pulse lengths, for example by better separation of the wanted signal from interference.

FIG. 2 also depicts act 130, in which the subject under examination 15 blocks the generated optical radiation to produce a shadow. In act 140, the shadow is detected by an optical detection unit 21.

Act 140 may be repeated successively, as depicted in FIG. 3. In the example, the shadows produced by the illumination of the subject under examination 15 by the radiation pulses 121, 122, 123, 124 are detected repeatedly. In the example, the times of the detection actions 140 are matched to the time windows of the radiation pulses 121, 122, 123, 124. Acts 120 and 140 are synchronized. For example, the time period T between the detection actions 140 equals the time period P between the illumination actions 121, 122, 123, 124.

At act 150, a position of the subject under examination 15 is determined from the shadow detected in act 140. It The position determination may involve using the information from not just one but a plurality of detection actions 140 to be able to also determine, for example, a position change and/or a motion.

FIG. 5 depicts a spatial arrangement of a radiation generating unit 19 and an optical detection unit 21 according to an embodiment. The radiation generating unit 19 includes four radiation sources 191, 192, 193, 194 that are arranged in different directions on different sides relative to the subject under examination 15. Such an arrangement provides for the generated optical radiation to illuminate the subject under examination from different directions.

For example, the generated optical radiation may illuminate the subject under examination successively from different directions. For example, as illustrated by FIG. 3, the radiation source 191 may emit a first radiation pulse 121 in direction D1, then the radiation source 192 may emit a second radiation pulse 122 in direction D2, then the radiation source 193 may emit a third radiation pulse 123 in direction D3, then the radiation source 194 may emit a fourth radiation pulse 124 in direction D4, and so on.

If the subject under examination 15, for example, a part of the subject under examination 15 includes a pointed contour, such as the nose 30, is illuminated, then as a result of the subject under examination 15 blocking the illumination, a region remains unilluminated, i.e. a shadow is produced behind the subject under examination 15. The pointed contour of the subject under examination 15 results in the shadow likewise including a pointed contour.

If, for example, the radiation source 191 illuminates the nose 30, then, from the perspective of the radiation source 191, a shadow 991 is produced behind the nose 30. FIG. 5 depicts a further shadow 992 that is produced by the radiation source 192 illuminating the nose 30.

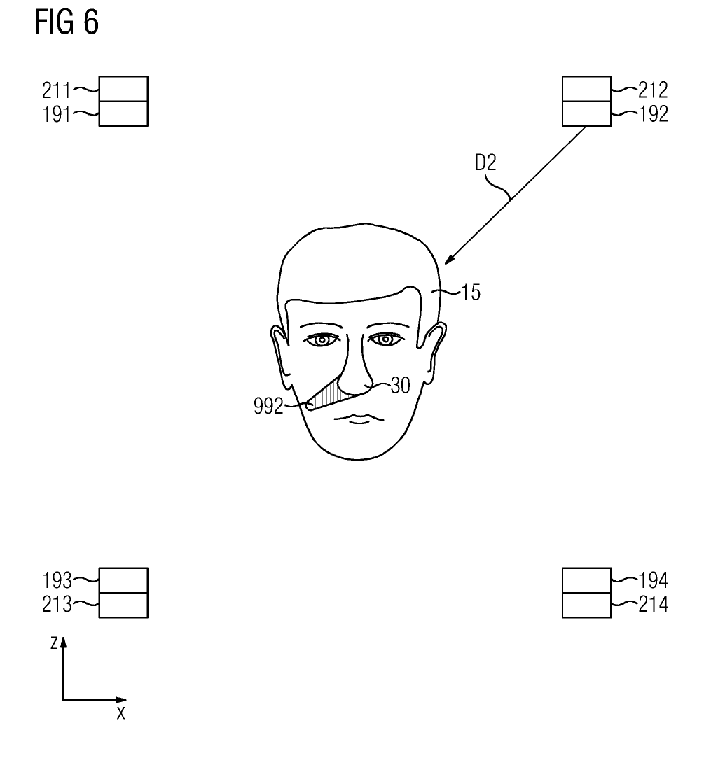

In an embodiment, the generated optical radiation illuminates the subject under examination 15 simultaneously from different directions. If, for example, the radiation sources 191 and 192 illuminate the nose 30 simultaneously, superposition of the illumination situations depicted in FIGS. 5 and 6, i.e. two shadows 991 and 992 are produced simultaneously. The contrast in brightness of the shadows 991 and 992 may be less strong than when the shadows are produced separately.

FIG. 5 depicts an example where the radiation generating unit 19 includes a radiation source 191 that is configured to emit the generated optical radiation in different directions. The direction D1 of the beam produced by the radiation source 191 may be rotated through an angle α into a direction D1α. The intensity of the shadow case by the illumination may be altered systematically.

The optical detection unit 21 includes four detection modules 211, 212, 213, 214 that are arranged on different sides relative to the subject under examination 15. The optical detection unit may detect the shadow 991, 992 from different directions. Each of the detection modules 211, 212, 213, 214 may also be arranged, as depicted, together with a respective radiation source 191, 192, 193, 194 in a shared housing.

The direction D1, D2, D3, D4 from which the generated optical radiation illuminates the subject under examination 15 may be determined from at least one already detected shadow 991, 992.

For example, if the position determination unit 13 identifies that the subject under examination 15 is moving, and the motion will result in deterioration of a shadow cast in future by illuminations from certain directions or even in unusable analysis data, then the system may forgo illumination from these directions. A suitable direction is selected instead.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims may, alternatively, be made to depend in the alternative from any preceding or following claim, whether independent or dependent, and that such new combinations are to be understood as forming a part of the present specification.

While the present invention has been described above by reference to various embodiments, it may be understood that many changes and modifications may be made to the described embodiments. It is therefore intended that the foregoing description be regarded as illustrative rather than limiting, and that it be understood that all equivalents and/or combinations of embodiments are intended to be included in this description.

1. A method for determining a position of a subject under examination during implementation of a medical imaging procedure, the method comprising:
generating, by a radiation generator, optical radiation;
illuminating, using the generated optical radiation, the subject under examination, wherein the subject under examination generates a shadow by blocking the generated optical radiation;
detecting, by an optical detector, the shadow; and
determining, by a position determiner, the position of the subject under examination based on the detected shadow.

2. The method of claim 1, wherein detecting the shadow is repeated successively.

3. The method of claim 1, wherein the optical radiation is pulsed by the radiation generating unit.

4. The method as claimed in claim 3, wherein the optical radiation is pulsed at a frequency of greater than 60 Hz.

5. The method of claim 1, wherein the shadow includes a pointed contour.

6. The method of claim 1, wherein the generated optical radiation illuminates the subject under examination from different directions.

7. The method of claim 6, wherein the generated optical radiation illuminates the subject under examination successively, simultaneously, or successively and simultaneously from the different directions.

8. The method of claim 6, wherein the generated optical radiation illuminates the subject under examination successively from the different directions.

9. The method of claim 1, wherein the radiation generating unit generates first optical radiation and second optical radiation,
wherein the subject under examination is illuminated from a first direction by the first generated optical radiation,
wherein the subject under examination is illuminated from a second direction by the second generated optical radiation,
wherein the first direction differs from the second direction,
wherein the first optical radiation has a first pulse frequency,
wherein the second optical radiation has a second pulse frequency, and
wherein the first pulse frequency differs from the second pulse frequency.

10. The method of claim 1, wherein the radiation generating unit generates first optical radiation and second optical radiation,
wherein the subject under examination is illuminated from a first direction by the first generated optical radiation,
wherein the subject under examination is illuminated from a second direction by the second generated optical radiation,
wherein the first direction differs from the second direction,
wherein the first optical radiation comprises pulses of a first pulse length,
wherein the second optical radiation comprises pulses of a second pulse length, and
wherein the first pulse length differs from the further pulse length.

11. The method of claim 1, wherein a direction from which the generated optical radiation illuminates the subject under examination is determined from at least one previously detected shadow.

12. The method of claim 1, wherein the radiation generating unit comprises a plurality of radiation sources that are arranged in different directions on different sides relative to the subject under examination.

13. The method of claim, 1, wherein the radiation generating unit comprises at least one radiation source configured to emit the generated optical radiation in different directions.

14. The method of claim 1, wherein the optical detection unit detects the shadow from different directions.

15. The method of claim 1, wherein the optical detection unit comprises a plurality of detection modules that are arranged on different sides relative to the subject under examination.

16. A medical imaging apparatus comprising:
a radiation generating unit configured to generate optical radiation;
an optical detection unit configured to detect a shadow produced as a result of a subject under examination blocking the generated optical radiation; and
a position determination unit configured to determine a position of the subject under examination as a function of the detected shadow.

17. The medical imaging apparatus of claim 16, wherein the radiation generating unit comprises at least one radiation source, and the detection unit comprises at least one detection module, and
wherein the at least one radiation source and the at least one detection module are arranged in a shared housing.

18. The medical imaging apparatus of claim 16, wherein the optical detection unit is configured to successively and repeatedly detect the shadow.

19. The medical imaging apparatus of claim 16, wherein the radiation generating unit is configured to generate pulsed optical radiation.

20. In a non-transitory computer readable storage medium that stores instructions executable by one or more processors to determine a position of a subject under examination during implementation of a medical imaging procedure, the instructions comprising:
generating optical radiation to illuminate the subject under examination, wherein the subject under examination blocks the generated optical radiation and produces a shadow;
detecting the shadow; and
determining the position of the subject under examination based on the detected shadow.
